(19) United States
(12) Patent Application Publication
(10) Pub. No.: US 2015/0368286 A1
(43) Pub. Date: Dec. 24, 2015

(54) METHODS OF PREPARING SUBSTITUTED NUCLEOTIDE ANALOGS

(71) Applicant: ALIOS BIOPHARMA, INC., South San Francisco, CA (US)

(72) Inventors: Vladimir Serebryany, Burlingame, CA (US); Leonid Beigelman, San Mateo, CA (US)

(21) Appl. No.: 14/746,219

(22) Filed: Jun. 22, 2015

Related U.S. Application Data
(60) Provisional application No. 62/016,229, filed on Jun. 24, 2014.

(51) Int. Cl. C07H 19/06; C07H 1/02; C07H 1/06; C07H 1/00

(52) U.S. Cl. CPC C07H 19/06; C07H 1/00; C07H 1/02; C07H 1/06; C07B 2200/13

(57) ABSTRACT

Disclosed herein are methods of preparing a phosphoroamidate nucleotide analog, which are useful in treating diseases and/or conditions such as viral infections.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6.

### BACKGROUND

1. Field

The present application relates to the fields of chemistry, biochemistry, and medicine. More particularly, disclosed herein are methods of preparing a phosphoroamidate nucleotide analog, which can be useful in treating diseases and/or conditions such as viral infections.

2. Description

Nucleoside analogs are a class of compounds that have been shown to exert antiviral and anticancer activity both in vitro and in vivo, and thus, have been the subject of widespread research for the treatment of viral infections and cancer. Nucleoside analogs are usually therapeutically inactive compounds that are converted by host or viral enzymes to their respective active anti-metabolites, which, in turn, may inhibit polymerases involved in viral or cell proliferation. The activation occurs by a variety of mechanisms, such as the addition of one or more phosphate groups and, or in combination with, other metabolic processes.

### SUMMARY

Some embodiments disclosed herein relate to a method of preparing compound (I), or a pharmaceutically acceptable salt thereof. Some embodiments disclosed herein relate to a method of preparing compound (I)(i) and/or compound (I)(ii), or a pharmaceutically acceptable salt of the foregoing. In some embodiments, a method described herein can provide compound (I), or a pharmaceutically acceptable salt thereof, that is diastereomerically enriched in compound (I)(ii), or a pharmaceutically acceptable salt thereof.

Other embodiments disclosed herein relate to Form A of compound (I).

Still other embodiments disclosed herein relate to a compound, or a pharmaceutically acceptable salt thereof, having the formula:

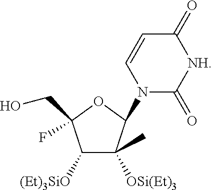

### BRIEF DESCRIPTION OF THE DRAWINGS

FIG. 1 is an XRPD spectrum of Form A.

FIG. 2 is a DSC and TGA spectrum of Form A.

FIG. 3 is a ³¹P NMR of compound (I) obtained from a method described herein.

### DETAILED DESCRIPTION

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), heteroaryl(alkyl), (heterocyclyl)alkyl, hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heterocyclyl group. That is, the alkyl, alkenyl, alkynyl, ring of the cycloalkyl, ring of the cycloalkenyl, ring of the aryl, ring of the heteroaryl or ring of the heterocyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heterocyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

The term “halogen atom” or “halogen” as used herein, means any one of the radio-stable atoms of column 7 of the Periodic Table of the Elements, such as, fluorine, chlorine, bromine and iodine.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

The term “crystalline” refers to a substance that has its atoms, molecules or ions packed in regularly ordered three-dimensional pattern. The term “substantially crystalline” refers to a substance where a substantial portion of the substance is crystalline. For example, substantially crystalline materials can have more than about 85% crystallinity (e.g., more than about 90% crystallinity, more than about 95% crystallinity, or more than about 99% crystallinity).

It is understood that the methods and combinations described herein include crystalline forms (also known as polymorphs, which include the different crystal packing arrangements of the same elemental composition of a compound), amorphous phases and salts.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

Likewise, it is understood that, in any compound described, all tautomeric forms are also intended to be included, for example, tautomers of heterocyclic bases known in the art are intended to be included, including tautomers of natural and non-natural purine-bases and pyrimidine-bases.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Where a range of values is provided, it is understood that the upper and lower limit, and each intervening value between the upper and lower limit of the range is encompassed within the embodiments.

Compound (I), or a pharmaceutically acceptable salt thereof, is active against HCV. Examples of methods for forming compound (I) are shown in Scheme 1.

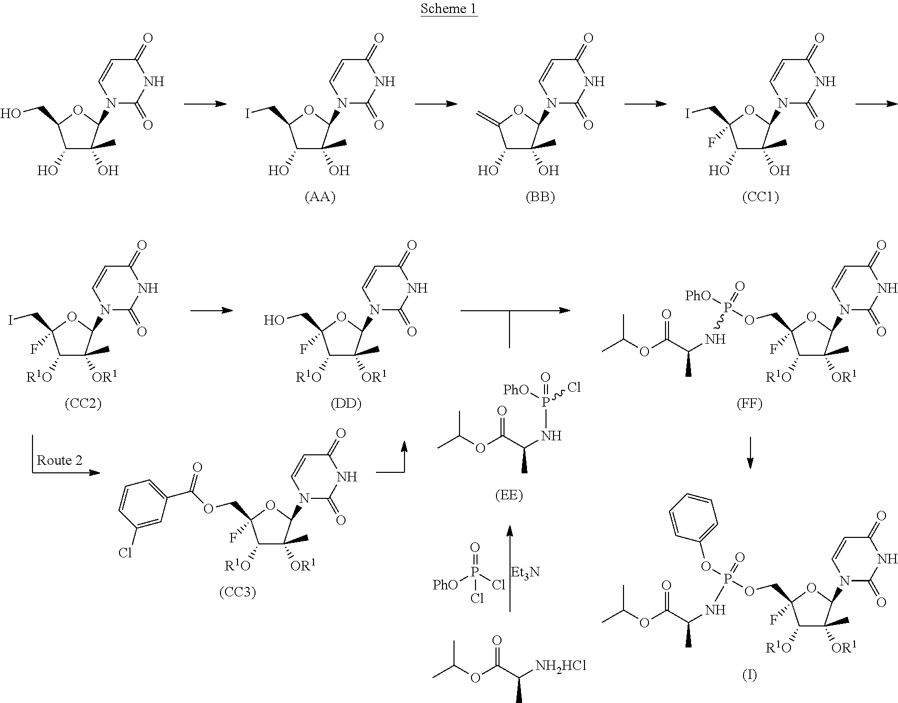

Some embodiments disclosed herein relate to a method of preparing a compound (I), or a pharmaceutically acceptable salt thereof, wherein the method can include the use of compound DD:

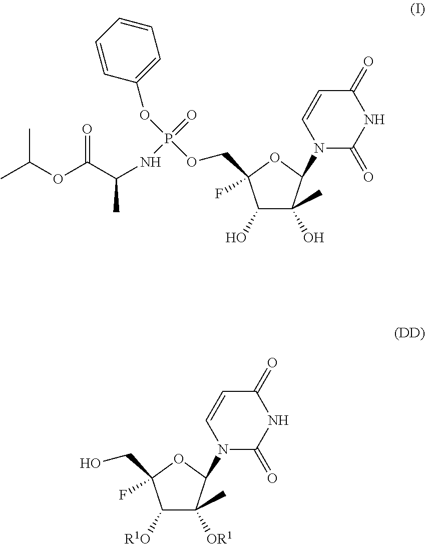

wherein each R¹ can be a silyl group.

Various silyl groups can be present on compound (DD). Examples of suitable silyl groups are described herein and include trimethylsilyl (TMS), triethylsilyl (TES), tert-butyldimethylsilyl (TBDMS), triisopropylsilyl (TIPS), tert-butyldiphenylsilyl (TBDPS), tri-iso-propylsilyloxymethyl and [2-(trimethylsilyl)ethoxy]methyl. In some embodiments, the R¹ groups can be the same. In other embodiments, the R¹ groups can be different. In some embodiments, the R¹ groups can both be triethylsilyl.

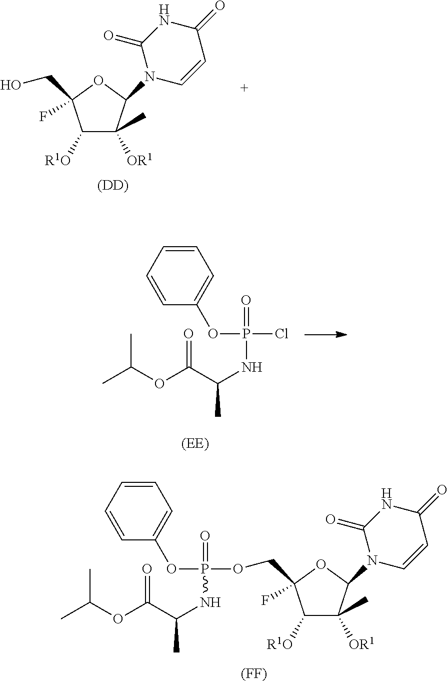

In some embodiments, a method described herein can include coupling compound (DD) and compound (EE) to form compound (FF). A variety of methods can be used in the reaction between compound (DD) and compound (EE). In some embodiments, compound (DD) can be coupled to compound (EE) using a base, an acid or a Grignard reagent. In some embodiments, to facilitate the coupling, a Grignard reagent can be used. Suitable Grignard reagents are known to those skilled in the art and include, but are not limited to, alkylmagnesium chlorides and alkylmagnesium bromides. In some embodiments, the Grignard reagent can have the general formula of R^{C}—MgBr or R^{C}—MgCl, wherein R^{C} can be an optionally substituted alkyl or an optionally substituted aryl. In some embodiments, a reaction between compound (DD) and compound (EE) can be conducted in the presence of a base. For example, compound (EE) can be added to a mixture of compound (DD) and a base. Examples of bases include, but are not limited to, an optionally substituted amine base, such as an alkylamine (including mono-, di- and tri-alkylamines (for example, monoethylamine, diethylamine and triethylamine)), optionally substituted pyridines (such as collidine) and optionally substituted imidazoles (for example, N-methylimidazole)). Additional examples of bases include inorganic bases, such as a hydroxide, a carbonate and a bicarbonate. In some embodiments, a reaction between compound (DD) and compound (EE) can be conducted in the presence of N-methylimidazole. In some embodiments, a reaction between compound (DD) and compound (EE) can be conducted in the presence of an acid. Example of a suitable acid is trifluoromethanesulfonic acid.

The coupling reaction between compound (DD) and compound (EE) can be conducted in a variety of solvent(s). In some embodiments, the solvent can be a polar aprotic solvent. Examples of polar aprotic solvents include, but are not limited to, dimethylformamide, tetrahydrofuran, ethyl acetate, acetone, acetonitrile, dimethyl sulfoxide or methyl isobutyl ketone. In some embodiments, the solvent can be tetrahydrofuran (THF).

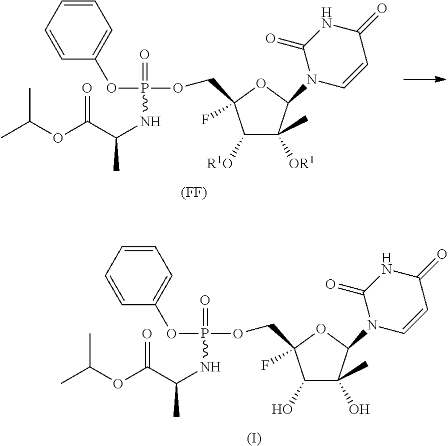

In some embodiments, a method described herein can include removing both R¹ groups from compound (FF) to obtain compound (I). A variety of methods and reagents can be used for removing the R¹ groups from compound (FF). For example, the R¹ groups can be removed under acidic conditions using an acid. Various suitable acids are known to those skilled in the art, such as hydrochloric acid, phosphoric acid, sulfuric acid and mixtures thereof. In some embodiments, the acid can be hydrochloric acid. The removal of both R¹ groups from compound (FF) to obtain compound (I) can be conducted in a solvent, for example, a polar aprotic solvent described herein. In some embodiments, the solvent used during the removal of the R¹ groups can be acetonitrile.

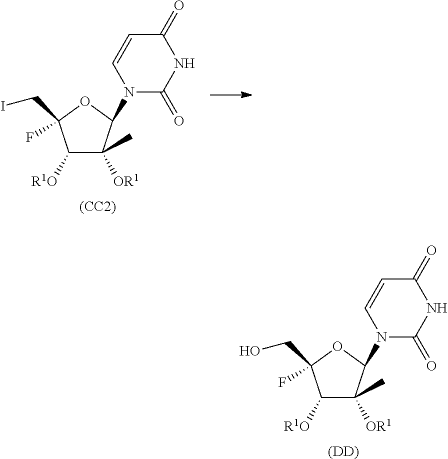

In some embodiments, a method described herein can include transforming compound (CC2) to compound (DD). An oxidant can be used in the conversation of the iodo group to a hydroxy group. An example of a suitable oxidant is a peracid, such as meta-chloroperoxybenzoic acid (mCPBA).

In some embodiments, compound (DD) can be obtained from compound (CC2) by converting the iodo group to a protected hydroxy group at the 5′-position of compound (CC2) and forming compound (CC3), wherein PG¹ can be a protecting group, followed by removal of the protecting group PG¹ under suitable conditions as described herein. The protected hydroxy group can be added to the 5′-carbon via a nucleophilic substitution reaction with an appropriate oxygen-containing nucleophile. When meta-chlorobenzoic acid (mCBA) is used as the oxygen-containing nucleophile, compound (CC3) can have the structure:

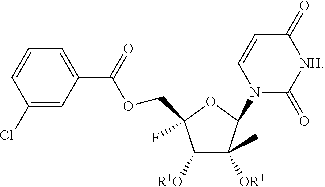

A tetralkylammonium salt can also be included when converting the iodo group to a protected hydroxy group at the 5′-position. Examples of suitable tetralkylammonium salts include, but are not limited to, tetrbutylammonium trifluoroacetic acid and tetrabutylammonium hydrogen sulfate. The protecting group, PG¹, can be removed using a variety of conditions. In some embodiments, the protected hydroxy group at the 5′-carbon can be removed via aminolysis using an amine base. Suitable amine bases are described herein. In some embodiments, the amine base can be n-butylamine. In some embodiments, the protecting group on the oxygen attached to the 5′-carbon can be removed using an inorganic base. Examples of suitable inorganic bases are described herein. In some embodiments, the inorganic base can be a hydroxide base, such as an alkali metal hydroxide base. In some embodiments, the hydroxide base can be sodium hydroxide.

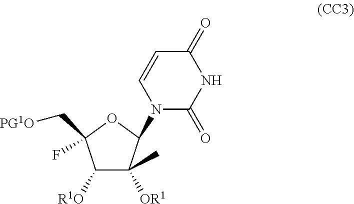

In some embodiments, compound (DD) can be obtained from compound (CC2) by using an oxidant, such as an oxidant described herein. An oxygen-containing nucleophile can displace the iodo group attached to the 5′-carbon via a nucleophilic substitution. The nucleophile can then be removed using suitable conditions to obtain compound (DD). For example, the nucleophile can be removed via hydrolysis. In some embodiments, the oxygen-containing nucleophile can be from a tetralkylammonium salt, such as those described herein, and the hydrolysis can be with water.

The protecting group, PG¹, can be removed via hydrolysis using a suitable base. Suitable bases are described herein. In some embodiments, the base can be an alkylamine (including mono-, di- and tri-alkylamines). For example, the alkylamine base can be monoethylamine, diethylamine, triethylamine and n-butylamine. In some embodiments, the base used to form compound (DD) from compound (CC3) selectively removes PG¹, and not the R¹ groups.

In some embodiments, compound (DD) can be crystallized using one or more solvents, such as polar aprotic solvents. Examples of polar aprotic solvents include, but are not limited to, dimethylformamide, tetrahydrofuran, ethyl acetate, acetone, acetonitrile, dimethyl sulfoxide or methyl isobutyl ketone. In some embodiments, the solvent can be tetrahydrofuran (THF). In some embodiments, the solvent can be acetonitrile. In some embodiments, the solvent can be a mixture of methyl isobutyl ketone and acetonitrile. Seed crystals of compound (DD) can be used to obtain compound (DD) if desired and/or needed.

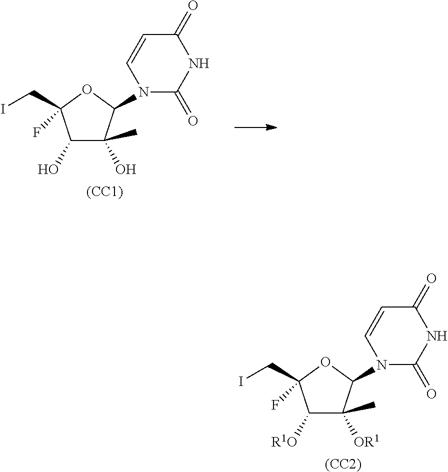

In some embodiments, a method described herein can include silylating compound (CC1) to form compound (CC2). Various compounds can be used to exchange the hydrogens of the 2′-OH and 3′-OH groups with silyl groups. In some embodiments, compound (CC1) can be silylated using a silyl halide. Examples of suitable silyl halides include silyl chlorides and silyl bromides. In some embodiments, the silyl halide can be a trialkylsilyl halide, triarylsilylhalide or alkyldiarylsilyl halide, such as a trialkylsilyl chloride and/or a trialkylsilyl bromide. If desired, the silylation can be catalyzed using a base. Examples of suitable bases are described herein and include an optionally substituted amine base, optionally substituted pyridines and optionally substituted imidazoles (for example). In some embodiments, the base can be an optionally substituted imidazole.

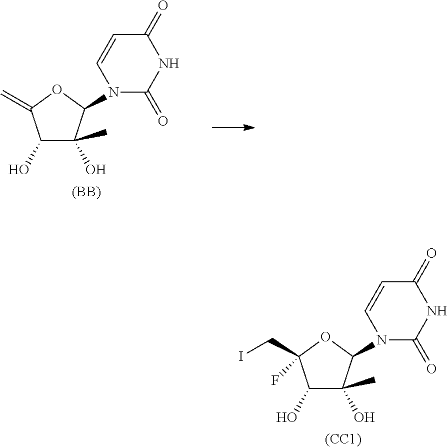

In some embodiments, a method described herein can include forming compound (CC1) from compound (BB) via an iodo-fluorination reaction. Suitable iodo sources are known to those skilled in the art. In some embodiments, the iodo source can be N-iodosuccinimide, iodine and/or iodine monochloride. Suitable fluoride sources are also known to those skilled in the art. In some embodiments, the fluoride source can be triethylamine.3HF, pyridine-HF and/or TBAF. The iodo source adds the iodo group to the 5′-position and the fluoride source adds the fluoro group to the 4′-position. The iodo-fluorination reaction can provide compound (CC1) in excess of the other diastereomer where the fluoro group is above the pentose ring. For example, compound (CC1) can be obtained in a ratio in the range of about 90 to about 10 (amount of compound (CC1)/amount of compound (CC1)+amount of other diastereomer). In some embodiments, compound (CC1) can be obtained in a ratio in the range of about 95 to about 5 (amount of compound (CC1)/amount of compound (CC1)+amount of other diastereomer).

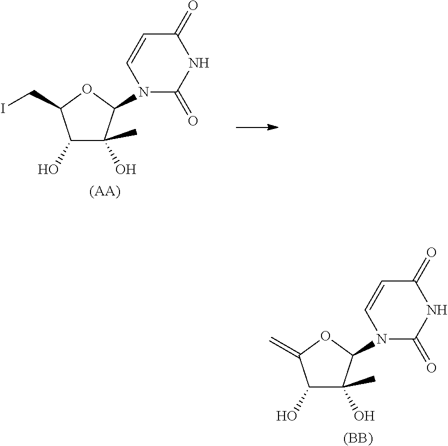

In some embodiments, a method described herein can include forming compound (BB) from compound (AA) via an elimination reaction. Methods and reagents for preparing compound (BB) from compound (AA) via an elimination reaction are known to those skilled in the art. In some embodiments, the elimination reaction can be conducted using a strong base. In some embodiments, the strong base can be selected from sodium methoxide, potassium hydroxide, sodium hydroxide and potassium ethoxide.

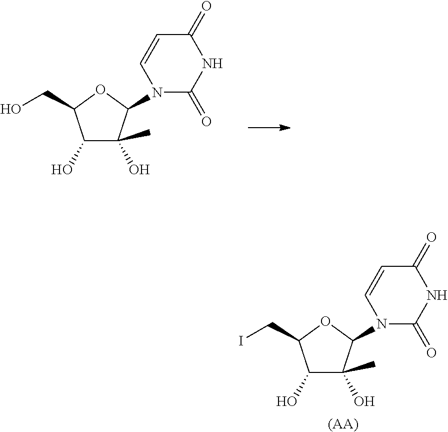

In some embodiments, a method described herein can include replacing the hydroxy group attached to the 5′-carbon of 2′-methyluridine with an iodo group to form compound (BB). The primary alcohol attached to the 5′-carbon of 2′-methyluridine can be converted to an iodoalkyl using an iodo source, a phosphine reagent and a base. In some embodiments, the iodo source can be I₂. Suitable phosphine reagents are known to those skilled in the art. In some embodiments, the phosphine reagent can be triphenylphosphine. Suitable bases that can be used in this conversion reaction from 2′-methyluridine to compound (AA) are described herein. In some embodiments, the base can be an optionally substituted imidazole.

In some embodiments, a method described herein can include crystallizing compound (I) from isopropyl acetate (IPAC). If desired and/or needed, seed crystals of compound (I) (for example, compound (I)(i) and/or compound (I)(ii)) can be added to the mixture of compound (I) and isopropyl acetate (IPAC).

In some embodiments, a method described herein can provide compound (I) that is a diastereomeric mixture of compound (I)(i) and compound (I)(ii), or a pharmaceutically acceptable salt of the foregoing:

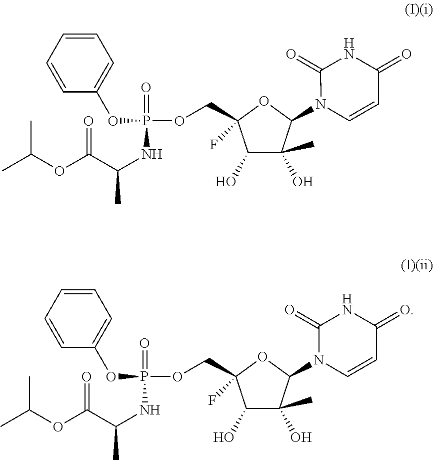 [SMILES: CC(C)OC(=O)[C@H](C)N[P@@](=O)(OC[C@@]1(F)O[C@@H](n2ccc(=O)[nH]c2=O)[C@](C)(O)[C@@H]1O)Oc1ccccc1 ; CC(C)OC(=O)[C@H](C)N[P@](=O)(OC[C@@]1(F)O[C@@H](n2ccc(=O)[nH]c2=O)[C@](C)(O)[C@@H]1O)Oc1ccccc1]

In some embodiments, a method described herein can include recrystallizing compound (I) from a mixture of an alcohol and a C₆₋₁₀ hydrocarbon. A variety of alcohols and C₆₋₁₀ hydrocarbons can be used for the recrystallization. In some embodiments, the alcohol can be ethanol. In some embodiments, the C₆₋₁₀ hydrocarbon can be selected from n-hexane and n-heptane. The amounts and ratio of alcohol to C₆₋₁₀ hydrocarbon can vary. In some embodiments, the ratio of alcohol to C₆₋₁₀ hydrocarbon can be in the range of about 1 to about 5 (alcohol:C₆₋₁₀ hydrocarbon). In some embodiments, the ratio of alcohol to C₆₋₁₀ hydrocarbon can be in the range of about 1 to about 4 (alcohol:C₆₋₁₀ hydrocarbon). In some embodiments, the ratio of alcohol to C₆₋₁₀ hydrocarbon can be in the range of about 1 to about 2 (alcohol:C₆₋₁₀ hydrocarbon).

In some embodiments, a method described herein can provide compound (I) that is diastereomerically enriched in compound (I)(ii). In some embodiments, the diastereomeric mixture of compound (I)(i) and compound (I)(ii) can be a diastereomeric mixture with a diastereomeric ratio of 1:5 or more of compound (I)(i) to compound (I)(ii) (compound (I)(i):compound (I)(ii)). In other embodiments, the diastereomeric mixture of compound (I)(i) and compound (I)(ii) can be a diastereomeric mixture with a diastereomeric ratio of 1:7 or more of compound (I)(i) to compound (I)(ii) (compound (I)(i):compound (I)(ii)). In still other embodiments, the diastereomeric mixture of compound (I)(i) and compound (I)(ii) can be a diastereomeric mixture with a diastereomeric ratio of 1:9 or more of compound (I)(i) to compound (I)(ii) (compound (I)(i):compound (I)(ii)). In yet still other embodiments, the diastereomeric mixture of compound (I)(i) and compound (I)(ii) can be a diastereomeric mixture with a diastereomeric ratio of 1:11 or more of compound (I)(i) to compound (I)(ii) (compound (I)(i):compound (I)(ii)). In some embodiments, the diastereomeric mixture of compound (I)(i) and compound (I)(ii) can be a diastereomeric mixture with a diastereomeric ratio of 1:13 or more of compound (I)(i) to compound (I)(ii) (compound (I)(i):compound (I)(ii)).

In some embodiments, compound (I) obtained from a method described herein can be diastereometrically enriched by >90% in compound (I)(ii) (eq. of compound (I)(ii)/(total eq. of compound (I)(i)+total eq. of compound (I)(ii)). In other embodiments, compound (I) obtained from a method described herein can be diastereometrically enriched by >95% in compound (I)(ii) (eq. of compound (I)(ii)/(total eq. of compound (I)(i)+total eq. of compound (I)(ii)). In still other embodiments, compound (I) obtained from a method described herein can be diastereometrically enriched by >98% in compound (I)(ii) (eq. of compound (I)(ii)/(total eq. of compound (I)(i)+total eq. of compound (I)(ii)). In yet still other embodiments, compound (I) obtained from a method described herein can be diastereometrically enriched by >99% in compound (I)(ii) (eq. of compound (I)(ii)/(total eq. of compound (I)(i)+total eq. of compound (I)(ii)).

In some embodiments, compound (I) obtained from the recrystallization can be more diastereomerically enriched in compound (I)(i) compared to the amount of diastereomeric enrichment of compound (I)(i) prior to recrystallization. In other embodiments, compound (I) obtained from the recrystallization can be more diastereomerically enriched in compound (I)(ii) compared to the amount of diastereomeric enrichment of compound (I)(ii) prior to recrystallization. In some embodiments, compound (I) obtained from the recrystallization can be more diastereomerically enriched in compound (I)(ii) compared to the amount of diastereomeric enrichment of compound (I)(ii) prior to recrystallization.

Some embodiments described herein generally related to a solid state form of compound (I), or a pharmaceutically acceptable salt thereof, for example a crystalline form of compound (I), or a pharmaceutically acceptable salt thereof. Some embodiments described herein generally related to a solid state form of compound (I)(ii), or a pharmaceutically acceptable salt thereof, for example a crystalline form of compound (I)(ii), or a pharmaceutically acceptable salt thereof.

### Form A

In some embodiments, compound (I) can be Form A of compound (I).

In some embodiments, Form A can be characterized by one or more peaks in an X-ray powder diffraction pattern, wherein the one or more peaks is selected from a peak in the range of from about 7.8 to about 8.6 degrees, a peak in the range of from about 10.2 to about 11.0 degrees, a peak in the range of from about 12.1 to about 12.9 degrees, a peak in the range of from about 16.2 to about 17.0 degrees, a peak in the range of from about 16.7 to about 17.5 degrees, a peak in the range of from about 17.0 to about 17.8 degrees, a peak in the range of from about 18.8 to about 19.6 degrees, a peak in the range of from about 19.2 to about 20.0 degrees, a peak in the range of from about 19.3 to about 20.1 degrees, a peak in the range of from about 19.9 to about 20.7 degrees, a peak in the range of from about 20.9 to about 21.7 degrees, and a peak in the range of from about 24.0 to about 24.8 degrees.

In some embodiments, Form A can be characterized by one or more peaks in an X-ray powder diffraction pattern, wherein the one or more peaks is selected from a peak at about 8.2 degrees, a peak at about 10.6 degrees, a peak at about 12.5 degrees, a peak at about 16.6 degrees, a peak at about 17.1 degrees, a peak at about 17.4 degrees, a peak at about 19.2 degrees, a peak at about 19.6 degrees, a peak at about 19.7 degrees, a peak at about 20.3 degrees, a peak at about 21.3 degrees and a peak at about 24.4 degrees.

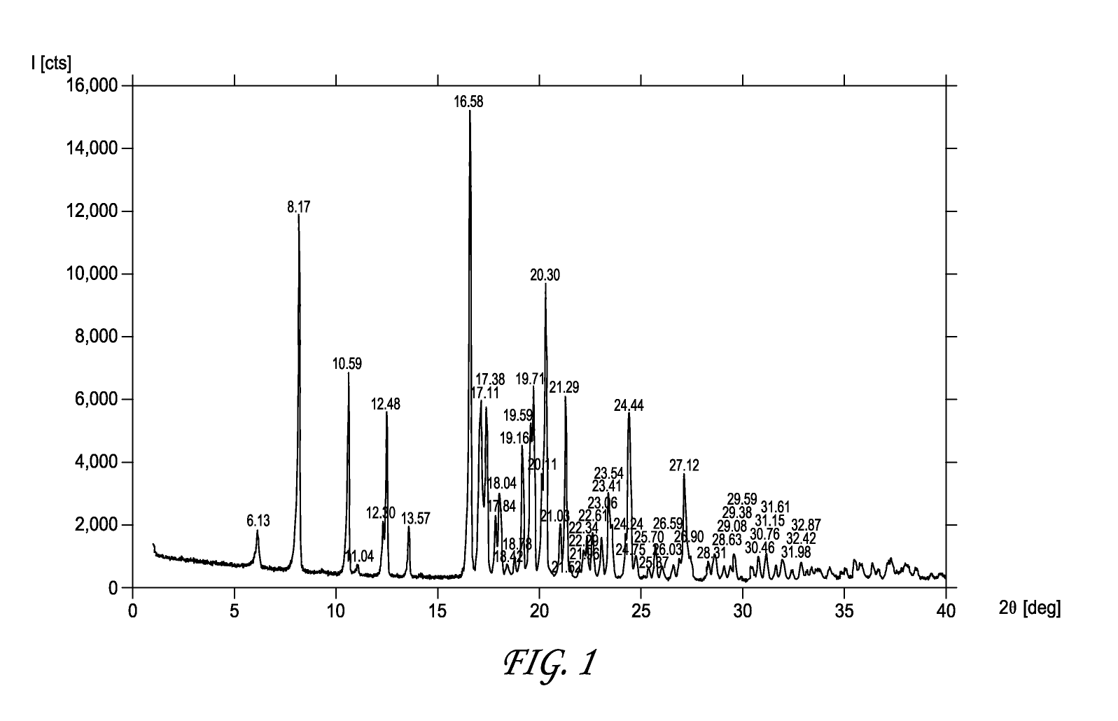

In some embodiments, Form A can exhibit an X-ray powder diffraction pattern as shown in FIG. 1. All XRPD spectra provided herein are measured on a degrees 2-Theta scale.

In some embodiments, Form A can be characterized by one or more peaks in an X-ray powder diffraction pattern selected from:

| No. | 2-Theta ° | Intensity % |
|---|---|---|
| 1 | 6.13* | 12 |
| 2 | 8.17* | 77 |
| 3 | 10.59* | 45 |
| 4 | 11.04 | 5 |
| 5 | 12.30* | 14 |
| 6 | 12.48* | 37 |
| 7 | 13.57* | 13 |
| 8 | 16.58* | 100 |
| 9 | 17.11* | 39 |
| 10 | 17.38* | 37 |
| 11 | 17.84* | 15 |
| 12 | 18.04* | 20 |
| 13 | 18.42 | 5 |
| 14 | 18.78 | 6 |
| 15 | 19.16* | 30 |
| 16 | 19.59* | 35 |
| 17 | 19.71* | 42 |
| 18 | 20.11* | 24 |
| 19 | 20.30* | 64 |
| 20 | 21.03* | 13 |
| 21 | 21.29* | 40 |
| 22 | 21.52 | 4 |
| 23 | 21.96 | 4 |
| 24 | 22.20 | 8 |
| 25 | 22.34* | 11 |
| 26 | 22.61* | 11 |
| 27 | 23.06* | 10 |
| 28 | 23.41* | 20 |
| 29 | 23.54* | 13 |
| 30 | 24.24* | 11 |
| 31 | 24.44* | 37 |
| 32 | 24.75 | 7 |
| 33 | 25.37 | 4 |
| 34 | 25.70 | 9 |
| 35 | 26.03 | 4 |
| 36 | 26.59 | 5 |
| 37 | 26.90 | 6 |
| 38 | 27.12* | 24 |
| 39 | 28.31 | 5 |
| 40 | 28.63 | 7 |
| 41 | 29.08 | 5 |
| 42 | 29.38 | 4 |
| 43 | 29.59 | 7 |
| 44 | 30.46 | 4 |
| 45 | 30.76 | 6 |
| 46 | 31.15 | 7 |
| 47 | 31.61 | 4 |
| 48 | 31.98 | 6 |

Peaks with an asterisk (*) are prominent peaks

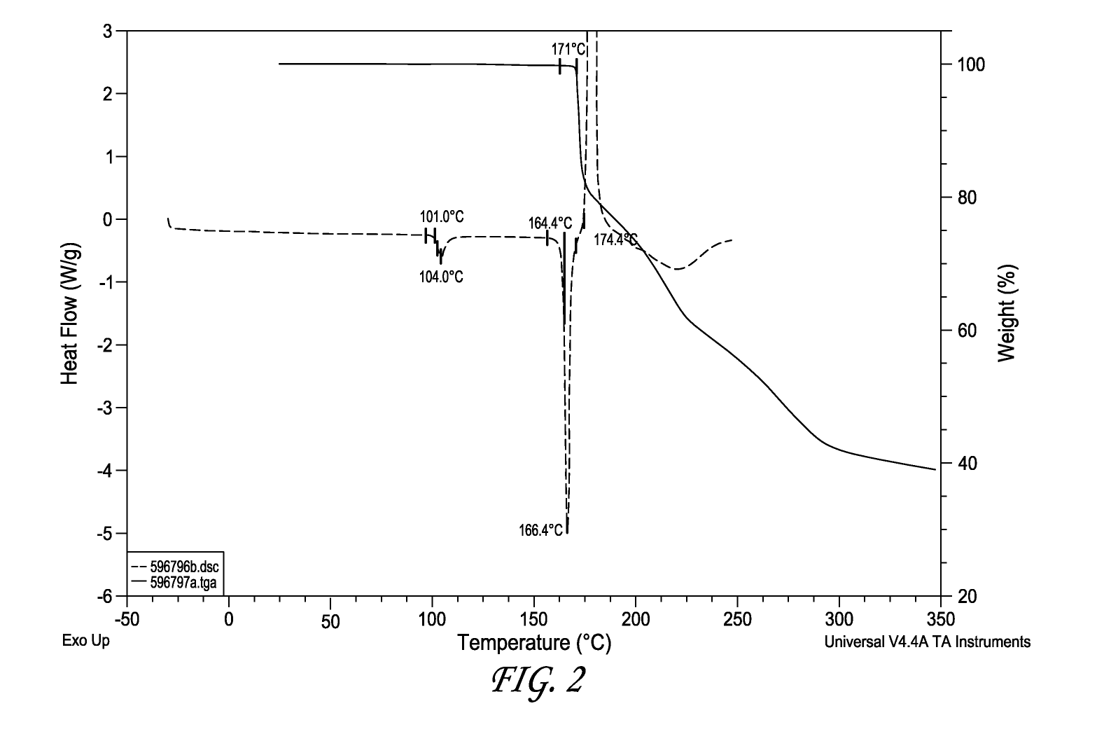

In some embodiments, Form A can be characterized by a DSC thermogram of FIG. 2. In some embodiments, Form A can be characterized by a first endoterm in the range of from about 95° C. to about 105° C. In other embodiments, Form A can be characterized by a first endoterm of about 104° C. In some embodiments, the first endoterm can correspond to a solid-solid transition from Form A to a second form of compound (I). In some embodiments, Form A can be characterized by a second endotherm in the range of from about 155° C. to about 175° C. In other embodiments, Form A can be characterized by a second endotherm of about 166° C. In some embodiments, Form A can be characterized by heat fluctuations starting at about 175° C. In some embodiments, the conversion of the second form of compound (I) to Form A can occur in the range of about 50° C. to about 65° C. In some embodiments, the conversion of the second form of compound (I) to Form A can occur at about 58° C. In some embodiments, compound (I) melts at a temperature in the range of from about 160° C. to about 170° C. In some embodiments, compound (I) melts at a temperature in the range of from about 164° C. to about 166° C. In some embodiments, compound (I) melts at about 166° C.

### EXAMPLES

Additional embodiments are disclosed in further detail in the following examples, which are not in any way intended to limit the scope of the claims.

### Example 1

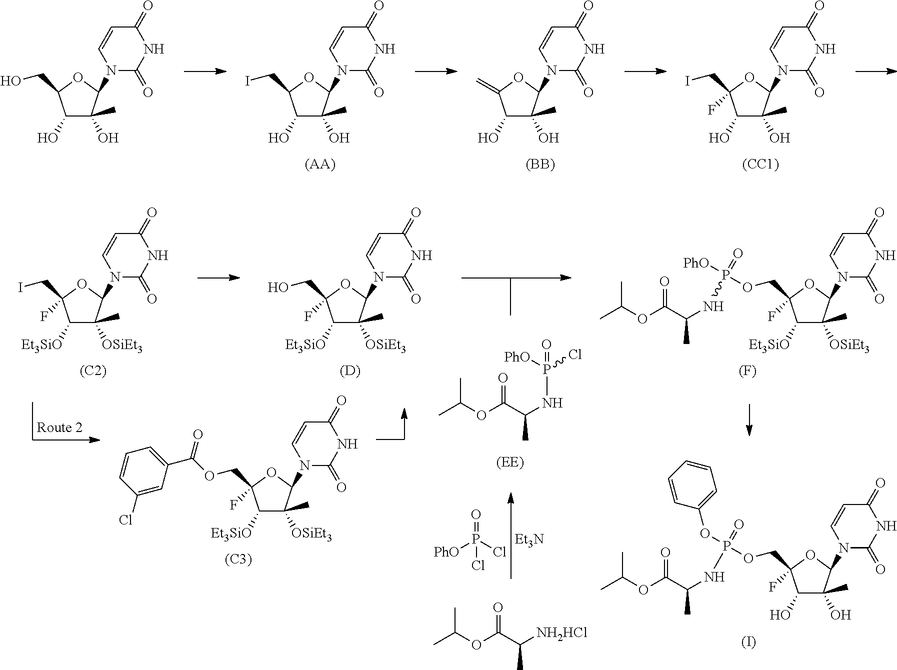

Abbreviations:

mCBA (meta-chlorobenzoic acid); mCPBA (meta-chloroperoxybenzoic acid); DCM (dichoromethane); DMF (dimethylformamide); 2-MeTHF (2-methyltetrahyrdofuran); MTBE (tert-butyl methyl ether); TFA (trifluoroacetic acid); ACN (acetonitrile); isopropyl acetate (IPAC).

Compound AA:

3-Neck 3 L flask was charged with 2′-methyluridine (129 g, 500 mmol, 1.0 eq.), triphenylphosphine (196.5 g, 750 mmol, 1.5 eq.), imidazole (51 g, 750 mmol, 1.5 eq.) and anhydrous THF (750 mL). With stirring under an argon atmosphere, iodine (143.4 g, 565 mmol, 1.13 eq.) was added as a solution in THF (˜300 mL), while maintaining the temperature below 25° C. The mixture was stirred overnight at room temperature (RT). THF was replaced by MeOH under reduced pressure. Compound AA precipitated from methanol. The solid was aged overnight at 0° C., filtered off, washed with cold MeOH and dried under reduced pressure at 45-50° C. to yield compound AA (114.6 g, 62%).

Compound BB:

To a suspension of compound AA (114.2 g, 310 mmol, 1 eq.) in MeOH (350 mL) was added sodium methoxide (176 mL 25% in MeOH, 775 mmol, 2.5 eq.). The mixture was heated at 60° C. for 3 h. HPLC showed complete conversion of Compound AA to compound BB. The mixture was cooled down to RT, and the excess of sodium methoxide was neutralized to pH ˜5-7 with acetic acid (˜30 mL) while maintaining the temperature below 25° C. Compound BB precipitated during the addition of acetic acid. The solid was aged overnight at 0° C., isolated by filtration, washed with cold MeOH and dried under reduced pressure at 45° C. to yield compound BB (60.9 g, 80.8%).

Compound CC1:

To a stirred at 0° C. slurry of compound BB (28.8 g, 120 mmol, 1.0 eq.) in CH₃CN (240 mL) was added Et₃N.3HF (9.77 mL, 60 mmol, 0.5 eq., 1.5 eq. of HF) followed by addition of N-iodosuccinimide (35.1 g, 156 mmol, 1.3 eq.). Cooling was removed, and the mixture was stirred at RT for 2 h. Compound CC1 precipitated. Compound CC1 was filtered off, washed with DCM until the filtrate became colorless (3×) and dried under vacuum to give compound CC1 (27.7 g, 59.8%) as a slightly yellow powder. The mother liquor (83% HPLC, 13% β-isomer) was concentrated under reduced pressure to an oil. The oil was diluted with DCM (˜100 mL). The solution was added to a stirred 10% aqueous solution of potassium bicarbonate (150 mL), followed by addition of sodium thiosulfate (˜5 g as pentahydrate). A precipitate formed. The precipitate was isolated by filtration, washed with water followed by cold IPA and dried under reduced pressure to yield a second crop of compound C (8.0 g, 17%). The overall yield of compound CC1 was (35.7 g, 76.8%).

Compound D, Route 1:

A solution of compound CC1 (30.88 g, 80 mmol, 1.0 eq.) and imidazole (19.0 g, 280 mmol, 3.5 eq.) in DMF (140 mL) was treated with chlorotriethylsilane (33.5 mL, 200 mmol, 2.5 eq.) while maintaining the temperature below 25° C. After overnight stirring, the mixture was taken into water (250 mL) and IPAC (250 mL). The organic phase was separated, washed with water and concentrated under reduced pressure to a yellowish solid, ˜59 g crude weight. A 3-neck 1-L flask was equipped with magnetic stirring bar, addition funnel and pH electrode. The flask was charged with tetrabutylammonium hydroxide (114 mL, 55% aqueous solution, 240 mmol, 3 eq.). With stirring, TFA (18.4 mL, 240 mmol, 3 eq.) was added slowly to pH 3.5 while maintaining the temperature below 20-25° C. Crude compound CC1 was added to the flask as a solution in DCM (250 mL). The mixture was stirred vigorously. mCPBA (99 g as 70%, 400 mmol, 5 eq.) was added portion-wise over ˜15 mins. The reaction temperature was maintained below 25° C. The mixture gradually became acidic (pH<1.5 in ˜1 h), and the pH was maintained between 1.8-2 by dropwise addition of 2N aqueous NaOH. After 6 h, the pH was brought to 3.5, and the mixture was stirred overnight (overall: 40 mL, 80 mmol, 1 eq. of NaOH).

The reaction was quenched by the addition of sodium thiosulphate (119 g as pentahydrate, 480 mmol, 1.2 eq. to mCPBA) while maintaining the temperature below 25° C. The mixture was subjected to reduced pressure to remove DCM. MTBE (˜200 mL) was added. The mixture was stirred for ˜10 mins. The mixture was then filtered, and the organic layer was separated. The aqueous phase was washed with MTBE (3×50 mL). The combined MTBE extracts were washed with 10% aqueous potassium bicarbonate (150 mL) followed by water. The organic solution was filtered through a silica gel plug (60 g, 15×95 mm), and additional MTBE (˜150 mL) was used to elute the compound. The combined organic solution was concentrated to a thick slurry (˜77 g, ˜40 mL MTBE) which was diluted with hexane (325 mL). The resulted slurry was stirred for 15 mins at reflux, cooled to RT and left at 0° C. overnight. Compound D (24.4 g, 60.5%) was isolated by filtration, washed with cold hexane and dried under reduced pressure. The mother liquor (˜20 g) was separated by column chromatography (350 g, step-wise gradient from 30 to 50% ethyl acetate-hexane). The desired fractions were concentrated, and compound D was isolated by crystallization from hexane (50 mL) to yield a second crop of compound D (3.3 g (8.2%).

Compound D, Route 2:

Compound CC1 (9.65 g, 25 mmol, 1.0 eq.) was silylated as described for Route 1 to furnish the crude bis-triethylsilyl ether (20 g). A 3-neck 250 mL flask, equipped with magnetic stirring bar and pH meter electrode was charged with tetrabutylammonium hydrogensulfate (9.3 g, 27.5 mmol, 1.1 eq.), di-potassium hydrogenphosphate (9.6 g, 55 mmol, 2.2 eq.), 3-clorobenzoic acid (4.3 g, 27.5 mmol, 1.1 eq.) and water (30 mL). The crude bis-triethylsilyl ether was added to the flask as a solution in DCM (60 mL). With stirring, mCPBA (27.7 g as 70%, 112.5 mmol, 4.5 eq.) was added portionwise over ˜5 mins. The reaction was stirred while maintaining the temperature below 25° C. The pH gradually decreased, and di-potassium hydrogenphosphate (4 g, 24 mmol, ˜1 eq) was used to maintain the pH at approx. 3.5-4.5. The mixture was stirred overnight.

Sodium sulfite (17 g, 135 mmol, 1.2 eq. to mCPBA) was added while maintaining the temperature below 25° C. A solution of potassium carbonate (10 g) in water (˜30 mL) was added to pH-8. A precipitate was filtered off and washed with DCM (˜50 mL). The biphasic filtrate was transferred to a separating funnel. The organic layer was separated, the aqueous layer was washed with DCM (3×15 mL). The combined organic solution was concentrated to a semi-crystalline residue, which was partitioned between IPAC (60 mL) and 10% potassium bicarbonate (50 mL). The organic layer was separated, washed with water and concentrated under reduced pressure to give a crystalline residue (18 g).

The crude compound was dissolved in n-butylamine (20 mL) using rotovap agitation under cooling. The solution was concentrated under vacuum, and the residue was dissolved in MTBE (˜50 mL). 2N Aqueous HCl was added to pH ˜2 (˜40 mL). The organic layer was separated, and washed sequentially with water, half-saturated sodium bicarbonate and water. MTBE was replaced with ACN under reduced pressure. The volume of the solution was adjusted to ˜60 mL with ACN. The solution was seeded with compound D crystals. The precipitated compound D was aged overnight at 0° C., isolated by filtration, washed with a small amount of cold ACN and dried under vacuum to give compound D (7.09 g, 55%). The mother liquor was separated by column chromatography (100 g, step-wise gradient from 25 to 50% ethyl acetate-hexane). The desired fractions were concentrated, and compound D was isolated by crystallization from hexane (˜30 mL) to yield a second crop of compound D (2.6 g, 20.6%).

Compound EE:

A cold (˜−70° C.) solution of phenyldichlorophosphate (29.7 mL, 200 mmol, 1 eq.) and L-alanine isopropyl ester hydrochloride (35 g, 210 mmol, 1.05 eq.) in anhydrous DCM (600 mL) was treated with triethylamine (54 mL, 420 mmol, 2.1 eq.) while maintaining the temperature below −40° C. The reaction was warmed to RT over ˜2 h and then stirred at RT for ˜1 h. The slurry was diluted with cyclohexane (500 mL). Precipitated triethylammonium hydrochloride was filtered off and washed with cyclohexane. The filtrate was concentrated under reduced pressure to ˜500 mL and passed through a silica gel pad (30 g, 65×15 mm). Additional cyclohexane (˜500 mL) was used to elute the compound from the silica gel. The filtrate was concentrated under reduced pressure to yield compound EE (51.4 g, 66.6% corrected) as an oil.

Compound F:

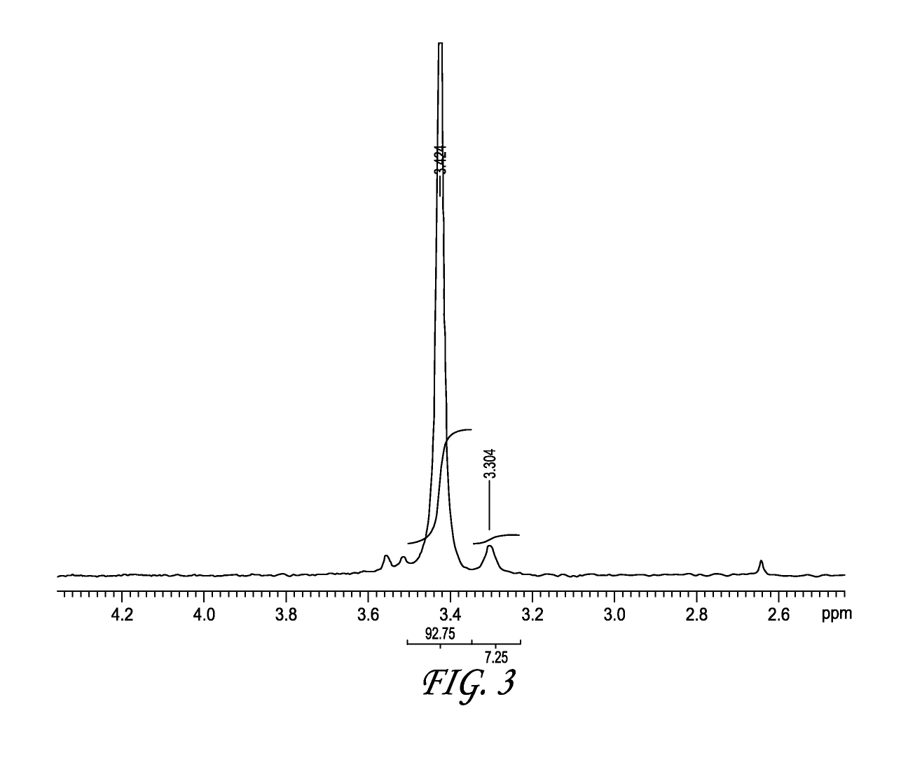

To a cold (−20° C.) solution of Compound D (28.0 g, 55.5 mmol, 1.0 eq.) in anhydrous THF (300 mL) was added iPrMgCl (2M in THF, 36 mL, 72 mmol, 1.3 eq.) dropwise while maintaining the temperature below −10-15° C. To this solution was added compound EE (42.5 g˜80%, 111 mmol, 2 eq.) as a solution in THF (˜20 mL). The mixture was warmed to 0° C. over 15 mins and then stirred at 0° C. The reaction product precipitated from the mixture. After 4 h, additional iPrMgCl (0.8 mL, 1.6 mmol, 0.03 eq.) was added. The mixture was left at 0-10° C. overnight. The reaction was quenched by sat. NH₄Cl (200 mL). The organic layer was separated, diluted with IPAC (˜200 mL) and washed with 10% aqueous potassium bicarbonate (200 mL). The organic layer was separated, washed with water and concentrated under reduced pressure to yield compound F an oil. ³¹P NMR of the crude product showed ˜93:7 mixture of (S_{P}):(R_{P}) diastereomers as shown in FIG. 3.

Compound (I):

The oil containing compound F was dissolved in anhydrous ACN (300 mL). The solution was treated with 4M HCl-dioxane (30 mL), and the reaction was allowed to proceed overnight at 0° C. The reaction was slowly poured into a stirred solution of aqueous potassium bicarbonate (250 mL 10%). After stirring for ˜15 mins, the organic layer was separated and concentrated under reduced pressure. The residue was dissolved in 2-MeTHF (˜300 mL). This solution was transferred back to the bicarbonate solution. The mixture was stirred for ˜1 h. The organic layer was separated and washed with diluted brine to neutral. The aqueous phases were back-extracted with 2-MeTHF. The combined organic solution was concentrated under reduced pressure, co-evaporated with IPAC. The crude residue (˜50 g) was dissolved in IPAC (˜100 mL). After polish filtration, the solution volume was adjusted to ˜150 mL with IPAC. Seeds of compound (I) crystals were added, and the crystallization mixture was slowly agitated for 5 h at RT. The precipitated solid of compound (I) was aged at 0° C. overnight, separated by filtration, washed with cold IPAC and dried under vacuum. Compound (I) (21.6 g, 71%) was obtained with 95% HPLC purity, (R_{P}) isomer 2.7%.

Recrystallization of Compound (I):

Approximately 95% pure compound (I) (25.3 g) was dissolved in EtOH (150 mL, reagent grade) at 60° C. The solution was polish filtered; and EtOH (˜50 mL) was used to rinse the glassware. The filtrate was slowly diluted with hexane (200 mL). The solution was seeded and allowed to cool to RT while being agitated slowly. The mixture was kept in a refrigerator overnight. The precipitated solid was filtered off, washed with a mixture of EtOH:hexane (1:2) and dried under vacuum. Purified compound (I) (22.2 g) was obtained with 99% HPLC purity; 0.7% (Rₚ)-diastereomer.

HPLC Conditions:
Column: Kinetex C18, 2.6μ, 150×4.6 mm (Phenomenex) Oven: 40° C.
Solvent A—water Solvent B—acetonitrile
Flow rate: 1 mL/min
Gradient: 5 to 95% B or 50 to 95% B (shown on each PDF) and 25 to 35% for the purity analysis of compound (I).

### Example 2

### Scaled-Up Procedure

The synthesis of compound (I) was scaled-up to a kilogram scale. Provided below are conditions that were modified in the scaled-up procedure.

### Compound AA:

| | | | Crystallization |
|---|---|---|---|
| | Ph₃P | Imidazole | Solvent |
| Example 1 | 1.5 eq. | 1.5 eq. | MeOH |
| Example 2 | 1.15 eq. | 1.2 eq. | EtOH |

Scaled-up Compound AA: 64.95 kg; Yield=76%; Purity=99.9% via HPLC.

### Compound BB:

Scaled-up Compound BB: 35.30 kg; Yield=82%; Purity=99.7% via HPLC.

### Compound CC1:

| | N-iodosuccinimide |
|---|---|
| Example 1 | 1.3 eq. |
| Example 2 | 1.4 eq. |
| | (1^{st} portion 1.3 eq.) |
| | (2^{nd} portion 0.1 eq.) |

Scaled-up Compound CC1: 30.9 kg; Yield=71%.

After the reaction was complete, 5-6 vol. of dichloromethane was added. The mixture was stirred for 2 h at 15-20° C. The mixture was then filtered, and the wet cake was rinsed with 2-3 vol. of dichloromethane. Yield=78.8%.

### Compound C2:

| | Et₃NSiCl | Work-up |
|---|---|---|
| Example 1 | 2.5 eq. | water |
| Example 2 | 3.0 eq. | 25% NaCl solution |

Compound C2 can be used in the next step. Compound C2 was also isolated by concentrating the solution of compound C2 in IPAC to 1-2 vol. n-Heptane (3×, 3.0-4.0 vol.) was added. The mixture was cooled to 0-5° C., and stirred at the same temperature for 7-8 h. The mixture was filtered and dried at 40-45° C. for 14-15 h. Compound C2 was obtained (29.0 kg, 90%, 99.6% purity via HPLC).

### Compound D, Route 2:

| | mCBA | mCPBA | Bu₄NHSO₄ |
|---|---|---|---|
| Example 1 | 1.1 eq. | 4.5 eq. | 1.1 eq. |
| Example 2 | 1.2 eq. | 5.0 eq. | 3.0 eq. |

Example 1 work-up: Seed crystals of compound D.

Example 2 work-up: Crude compound D was dissolved in DCM (1-2 vol.). N-heptane was added (3.0-6.0 vol.) and the temperature was adjusted to 15-20° C. The mixture was stirred for 5-6 h. The mixture was then filtered, and the filter cake was washed with DCM:n-heptane (v:v, 1:5). After drying for 14-15 h at 40-45° C., compound D (42.6 kg, 45%) was obtained.

NaOH and EtOH were used in place of n-BuNH₂, and column chromatography was not performed. Yield=69.7%.

### Compound F:

Scaled-up Compound F: 32.8 kg.

### Recrystallization of Compound (I):

| | Solvent | Temp. |
|---|---|---|
| Example 1 | n-hexane | 60° C. |
| Example 2 | n-heptane | 45-50° C. |

Example 2 crystallization: EtOH (7.0-8.0 vol.) and compound (I) were combined. The mixture was heated to 45-50° C. The mixture was then filtered and washed with ethanol (0.5-1.0 vol.) while maintaining the temperature at 45-50° C. At this same temperature, n-heptane (8.0 vol.) was charged in portions. The mixture was stirred 1-2 h at 45-50° C. The temperature was adjusted to 0-5° C., and the mixture was stirred 5-8 h. The mixture was then filtered and the filtrate was washed with EtOH:n-heptane (v:v, 1:2). After drying for 14-15 h at 40-45° C., compound (I)(ii) (2.1 kg, 32%, 98.8% purity via HPLC) was obtained.

X-Ray Powder Diffraction (XRPD)—Transmission Mode:

XRPD patterns were collected with a PANalytical X'Pert PRO MPD diffractometer using an incident beam of Cu radiation produced using an Optix long, fine-focus source. An elliptically graded multilayer mirror was used to focus Cu Kα X-ray radiation through the specimen and onto the detector. Prior to the analysis, a silicon specimen (NIST SRM 640d) was analyzed to verify the observed position of the Si (111) peak is consistent with the NIST-certified position. A specimen of the sample was sandwiched between 3-μm-thick films and analyzed in transmission geometry. A beam-stop, short antiscatter extension, and antiscatter knife edge, were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b. The XRPD pattern is shown in FIG. 1.

Differential Scanning Calorimetry (DSC):

DSC analyses were performed using a TA Instruments 2920 and Q2000 differential scanning calorimeters. Temperature calibration was performed using NIST-traceable indium metal. The sample was placed into an aluminum DSC pan, covered with a lid, and the weight was accurately recorded. A weighed aluminum pan (T0C=Tzero crimped pan) configured as the sample pan was placed on the reference side of the cell. The sample was heated at 10° C./min. The sample was then cooled to ambient temperature with 1° C./min rate. The DSC data shown in FIG. 2.

Thermogravimetry (TGA):

TG analyses were performed using a TA Instruments 2950 thermogravimetric analyzer. Temperature calibration was performed using nickel and Alumel™. Each sample was placed in an aluminum pan and inserted into the TG furnace. The furnace was heated under a nitrogen purge. The TGA data is provided in FIG. 2.

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

What is claimed is:

1. A method of preparing a compound (I), or a pharmaceutically acceptable salt thereof, wherein the method comprises the use of compound DD, wherein compound (I) and compound (DD) have the following structures: 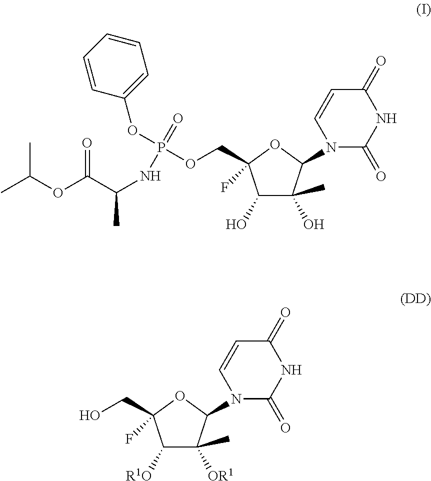
wherein:
each R¹ is a silyl group.

2. The method of claim 1, wherein each silyl group is selected from the group consisting of trimethylsilyl (TMS), triethylsilyl (TES), tert-butyldimethylsilyl (TBDMS), triisopropylsilyl (TIPS), tert-butyldiphenylsilyl (TBDPS), tri-iso-propylsilyloxymethyl and [2-(trimethylsilyl)ethoxy]methyl.

3. The method of claim 1, wherein both silyl groups are a triethylsilyl (TES) group.

4. The method of claim 1, comprising coupling compound DD and compound EE to form compound (FF):

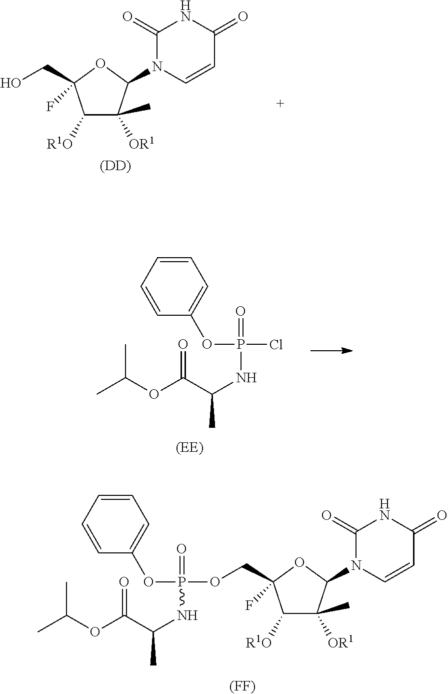

5. The method of claim 4, wherein the coupling is performed in the presence of a base, an acid or a Grignard reagent.

6. The method of claim 5, wherein the Grignard reagent is an optionally substituted alkylmagnesium chloride or an optionally substituted alkylmagnesium bromide.

7. The method of claim 5, wherein Grignard reagent has the formula of R^{C}—MgBr or R^{C}—MgCl, wherein R^{C} can be an optionally substituted alkyl or an optionally substituted aryl.

8. The method of claim 4, wherein the coupling reaction is conducted in a polar aprotic solvent.

9. The method of claim 8, wherein the solvent is tetrahydrofuran (THF).

10. The method of claim 4, further comprising removing both R¹ groups from compound (FF) to obtain compound (I):

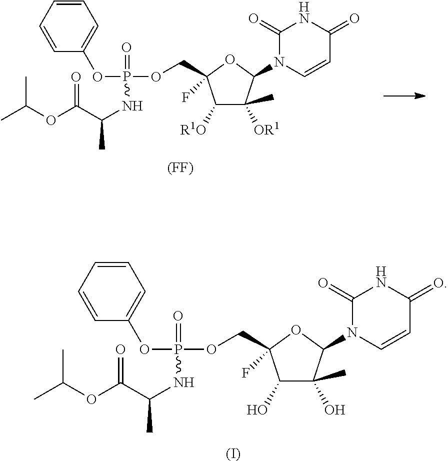

11. The method of claim 1, wherein compound (I) comprises a diastereomeric mixture of compound (I)(i) and compound (I)(ii),

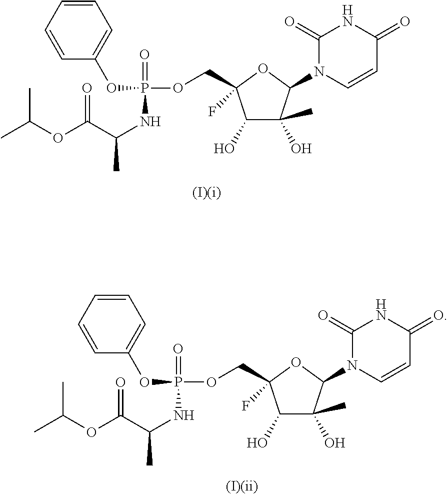 [SMILES: CC(C)OC(=O)[C@H](C)N[P@@](=O)(OC[C@@]1(F)O[C@@H](n2ccc(=O)[nH]c2=O)[C@](C)(O)[C@@H]1O)Oc1ccccc1 ; "(I)(i)" ; CC(C)OC(=O)[C@H](C)N[P@](=O)(OC[C@@]1(F)O[C@@H](n2ccc(=O)[nH]c2=O)[C@](C)(O)[C@@H]1O)Oc1ccccc1 ; "(I)(ii)"]

12. The method of claim 10, wherein the method further comprises recrystallizing compound (I) from a mixture of an alcohol and a C₆₋₁₀ hydrocarbon.

13. The method of claim 12, wherein the alcohol is ethanol.

14. The method of claim 12, wherein the C₆₋₁₀ hydrocarbon is selected from the group consisting of n-hexane and n-heptane.

15. The method of claim 12, wherein the mixture is in a ratio of alcohol to C₆₋₁₀ hydrocarbon in the range of about 1 to about 5 (alcohol:C₆₋₁₀ hydrocarbon).

16. The method of claim 11, wherein the diastereomeric mixture of compound (I)(i) and compound (I)(ii) is diastereomerically enriched in compound (I)(ii).

17. The method of claim 16, wherein the diastereomeric mixture of compound (I)(i) and compound (I)(ii) is a diastereomeric mixture with a diastereomeric ratio of 1:5 or more of compound (I)(i) to compound (I)(ii) (compound (I)(i):compound (I)(ii)).

18. The method of claim 16, wherein the diastereomeric mixture of compound (I)(i) and compound (I)(ii) is a diastereomeric mixture with a diastereomeric ratio of 1:7 or more of compound (I)(i) to compound (I)(ii) (compound (I)(i):compound (I)(ii)).

19. The method of claim 16, wherein the diastereomeric mixture of compound (I)(i) and compound (I)(ii) is a diastereomeric mixture with a diastereomeric ratio of 1:9 or more of compound (I)(i) to compound (I)(ii) (compound (I)(i):compound (I)(ii)).

20. The method of claim 16, wherein the diastereomeric mixture of compound (I)(i) and compound (I)(ii) is a diastereomeric mixture with a diastereomeric ratio of 1:11 or more of compound (I)(i) to compound (I)(ii) (compound (I)(i):compound (I)(ii)).

21. The method of claim 16, wherein the diastereomeric mixture of compound (I)(i) and compound (I)(ii) is a diastereomeric mixture with a diastereomeric ratio of 1:13 or more of compound (I)(i) to compound (I)(ii) (compound (I)(i):compound (I)(ii)).

22. The method of claim 16, wherein compound (I) is diastereometrically enriched by >90% in compound (I)(ii) (eq. of compound (I)(ii)/(total eq. of compound (I)(i)+total eq. of compound (I)(ii)).

23. The method of claim 16, wherein compound (I) is diastereometrically enriched by >95% in compound (I)(ii) (eq. of compound (I)(ii)/(total eq. of compound (I)(i)+total eq. of compound (I)(ii)).

24. The method of claim 16, wherein compound (I) is diastereometrically enriched by >98% in compound (I)(ii) (eq. of compound (I)(ii)/(total eq. of compound (I)(i)+total eq. of compound (I)(ii)).

25. The method of claim 16, wherein compound (I) is diastereometrically enriched by >99% in compound (I)(ii) (eq. of compound (I)(ii)/(total eq. of compound (I)(i)+total eq. of compound (I)(ii)).

26. The method of claim 1, further comprising crystallizing compound (I) from isopropyl acetate (IPAC).

27. The method of claim 1, further comprising transforming compound (CC2) to compound (DD):

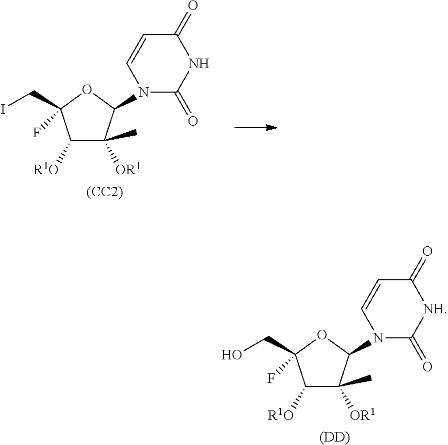

28. The method of claim 27, further comprising silylating compound (CC1) to form compound (CC2):

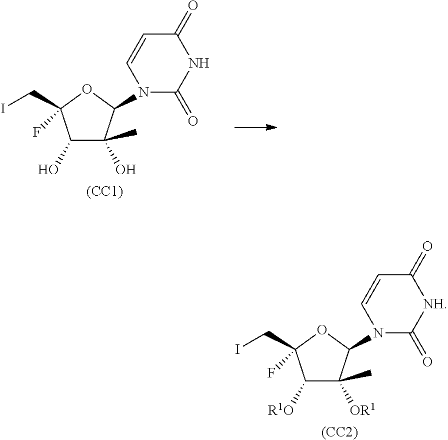

29. The method of claim 28, wherein compound (CC1) is silylated using a silyl halide.

30. The method of claim 29, wherein the silyl halide is silyl chloride.

31. The method of claim 29, wherein the silyl halide is trialkylsilyl halide.

32. The method of claim 28, further comprising forming compound (CC1) from compound (BB) via an iodo-fluorination reaction:

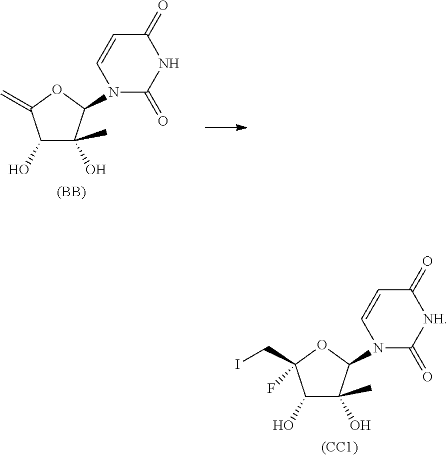

33. The method of claim 32, further comprising forming compound (BB) from compound (AA) via an elimination reaction:

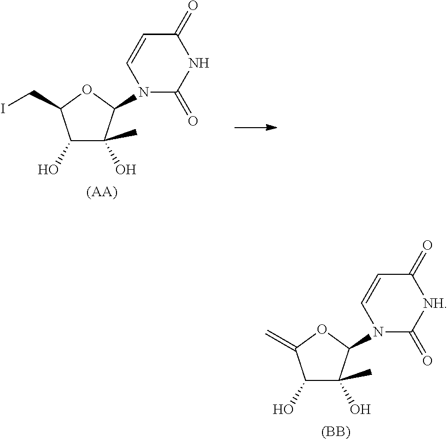

34. The method of claim 33, further comprising replacing the hydroxy group attached to the 5 ‘-carbon of 2’-methyluridine with an iodo group to form compound (BB):

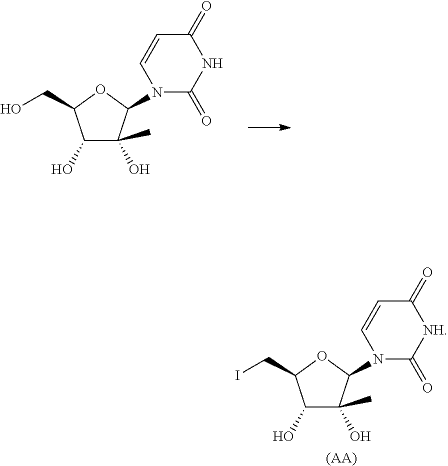

35. A compound, or a pharmaceutically acceptable salt thereof, having the formula:

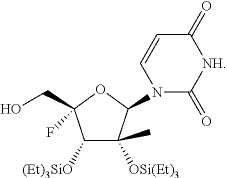

36. Form A of compound (I).

37. Form A of claim 36, wherein Form A is characterized by one or more peaks in an X-ray powder diffraction pattern, wherein the one or more peaks is selected from a peak in the range of from about 7.8 to about 8.6 degrees, a peak in the range of from about 10.2 to about 11.0 degrees, a peak in the range of from about 12.1 to about 12.9 degrees, a peak in the range of from about 16.2 to about 17.0 degrees, a peak in the range of from about 16.7 to about 17.5 degrees, a peak in the range of from about 17.0 to about 17.8 degrees, a peak in the range of from about 18.8 to about 19.6 degrees, a peak in the range of from about 19.2 to about 20.0 degrees, a peak in the range of from about 19.3 to about 20.1 degrees, a peak in the range of from about 19.9 to about 20.7 degrees, a peak in the range of from about 20.9 to about 21.7 degrees, and a peak in the range of from about 24.0 to about 24.8 degrees.

38. Form A of claim 36, wherein Form A is characterized by one or more peaks in an X-ray powder diffraction pattern, wherein the one or more peaks is selected from a peak at about 8.2 degrees, a peak at about 10.6 degrees, a peak at about 12.5 degrees, a peak at about 16.6 degrees, a peak at about 17.1 degrees, a peak at about 17.4 degrees, a peak at about 19.2 degrees, a peak at about 19.6 degrees, a peak at about 19.7 degrees, a peak at about 20.3 degrees, a peak at about 21.3 degrees and a peak at about 24.4 degrees.

39. Form A of claim 36, wherein Form A exhibits an X-ray powder diffraction pattern as shown in FIG. 1.

40. Form A of claim 36, wherein Form A is characterized by one or more peaks in an X-ray powder diffraction pattern selected from:

| No. | 2-Theta ° |
|---|---|
| 1 | 6.13* |
| 2 | 8.17* |
| 3 | 10.59* |
| 4 | 11.04 |
| 5 | 12.30* |
| 6 | 12.48* |
| 7 | 13.57* |
| 8 | 16.58* |
| 9 | 17.11* |
| 10 | 17.38* |
| 11 | 17.84* |
| 12 | 18.04* |
| 13 | 18.42 |
| 14 | 18.78 |
| 15 | 19.16* |
| 16 | 19.59* |
| 17 | 19.71* |
| 18 | 20.11* |
| 19 | 20.30* |
| 20 | 21.03* |
| 21 | 21.29* |
| 22 | 21.52 |
| 23 | 21.96 |
| 24 | 22.20 |
| 25 | 22.34* |
| 26 | 22.61* |
| 27 | 23.06* |
| 28 | 23.41* |
| 29 | 23.54* |
| 30 | 24.24* |
| 31 | 24.44* |
| 32 | 24.75 |
| 33 | 25.37 |
| 34 | 25.70 |
| 35 | 26.03 |
| 36 | 26.59 |
| 37 | 26.90 |
| 38 | 27.12* |
| 39 | 28.31 |
| 40 | 28.63 |
| 41 | 29.08 |
| 42 | 29.38 |
| 43 | 29.59 |
| 44 | 30.46 |
| 45 | 30.76 |
| 46 | 31.15 |
| 47 | 31.61 |
| 48 | 31.98 |

41. Form A of claim 36, wherein Form A is characterized by a DSC thermogram as shown in FIG. 2.

42. Form A of claim 41, wherein Form A is characterized by a first endoterm in the range of from about 95° C. to about 105° C.

43. Form A of claim 41, wherein Form A is characterized by a second endotherm in the range of from about 155° C. to about 175° C.

44. Form A of claim 41, wherein Form A is characterized by heat fluctuations starting at about 175° C.
